(19) United States
(12) Patent Application Publication
(10) Pub. No.: US 2009/0194107 A1
(43) Pub. Date: Aug. 6, 2009

(54) Mini-nasal mask

(76) Inventors: Edward A. Loeser, Sandy, UT (US); Jerold B. Martin, Midvale, UT (US); Bob Powers, SR., Englewood, CO (US)

Correspondence Address: TRASKBRITT, P.C., P.O. BOX 2550, SALT LAKE CITY, UT, 84110, US

(21) Appl. No.: 12/012,639

(22) Filed: Feb. 4, 2008

(51) Int. Cl. A61M 15/00; A61M 16/00; A62B 7/10; A61B 19/00

(52) U.S. Cl. 128/204.11; 128/206.11; 128/206.13; 128/206.14; 128/898

(57) ABSTRACT

A mini-nasal filter including a filter element and an anchoring arrangement adapted to hold the filter element in registration external to, and under, a user's nose. An operable anchoring arrangement includes one or more section of adhesive tape that can be affixed to the user's nose, and/or lip. The filter element may carry a masking scent, or be impregnated with a substance to enhance adherence of particulate matter to the filter. Certain masks may include a bioactive agent to resist germs, bacteria, and viruses, and/or a treatment compound for nasal therapy.

### BACKGROUND OF THE INVENTION

1. The Field of the Invention

The present invention relates to personal air filtration systems.

2. Related Art

There are many situations in which an unobtrusive personal air filtration system would be advantageous. For example, airplane passengers and attendants are exposed to unfiltered germs in a closely confined space. Yet, they are constrained by consideration of appearance, social norms, work regulations, ease of consuming food and beverages, and ease of communication (e.g. both verbal and lip reading), from wearing a bulky face-covering mask or respiratory device. Known intranasal filtration devices are hard to keep in place, uncomfortable (they distend the nares), can cause nose bleeds, and are generally irritating.

United States utility patents related to nasal filtering or therapy include: U.S. Pat. Nos. 4,267,831; 4,706,663; 5,746,200; 5,890,491; 5,993,716; 6,058,931; 6,119,690; 6,561,188; 6,752,149; 6,971,387; 7,108,198; and 7,156,098. United States design patents related to nasal filtration or therapy include D401323 and D451193.

It would be an improvement to provide an unobtrusive nasal air filter to provide filtration of inspired air while not disfiguring an individual's appearance, interfering with eating or drinking, distorting speech, blocking certain facial expressions, and/or interfering with lip reading.

### BRIEF SUMMARY

The invention provides a user with a personal nasal mask including a filter element structured for disposition at, and external to, the intake area of the nostrils of a human nose. The filter element is desirably sized such that it can be entirely disposed within a boundary sized in general agreement with a bottom of the nose. Typically, a perimeter of the filter element is smaller in size than a 1½ inch square. Sometimes, a filter element carries an aromatic cover scent. Certain filter elements may carry a bioactive agent and/or a treatment compound for nasal therapy. A filter element may be impregnated with a substance effective to enhance adherence of particles onto a surface of the filter element.

Desirably, a filter element is configured to provide an enlarged filtering surface area compared to the area defined by the nostril entrances. One operable filtering surface area is larger, by a factor greater than about 1.2, than the area of a surface that would be defined by a membrane stretched from the boundary perimeter of the filtering surface area. One filter element within contemplation is structured to permit a user to modify a size of the filtering surface area prior to installation of the mask.

In any case, the filter element is secured in place beneath the user's nose by an anchor arrangement. A currently preferred anchor arrangement includes a length of adhesive tape that is structured to permit its attachment to an external surface of the nose, lip, and/or other facial structure, such as a portion of the cheek. Desirably, the adhesive tape is substantially transparent or flesh-colored, to reduce its visible impact. However, in certain cases, the mask may be structured to provide a striking and/or noticeable appearance. The adhesive tape may sometimes be formed as an integral perimeter portion of the filter element. Another operable anchor arrangement includes an elastic strap structured for disposition to encircle a user's head, or arranged for attachment to a user's ear.

### BRIEF DESCRIPTION OF THE FIGURES

FIG. 1 is a side view of a human nose;

FIG. 2 is a side view of the nose illustrated in FIG. 1, with a mini-nasal mask installed thereon;

FIG. 3 is a plan view of a nasal mask structured according to certain principles of the instant invention;

FIG. 4 is a view in elevation of a cross-section taken through an alternative nasal mask;

FIG. 5 is a view in elevation of a cross-section taken through another alternative nasal mask; and

FIG. 6 is a side view of an alternative nasal mask installed on a human face.

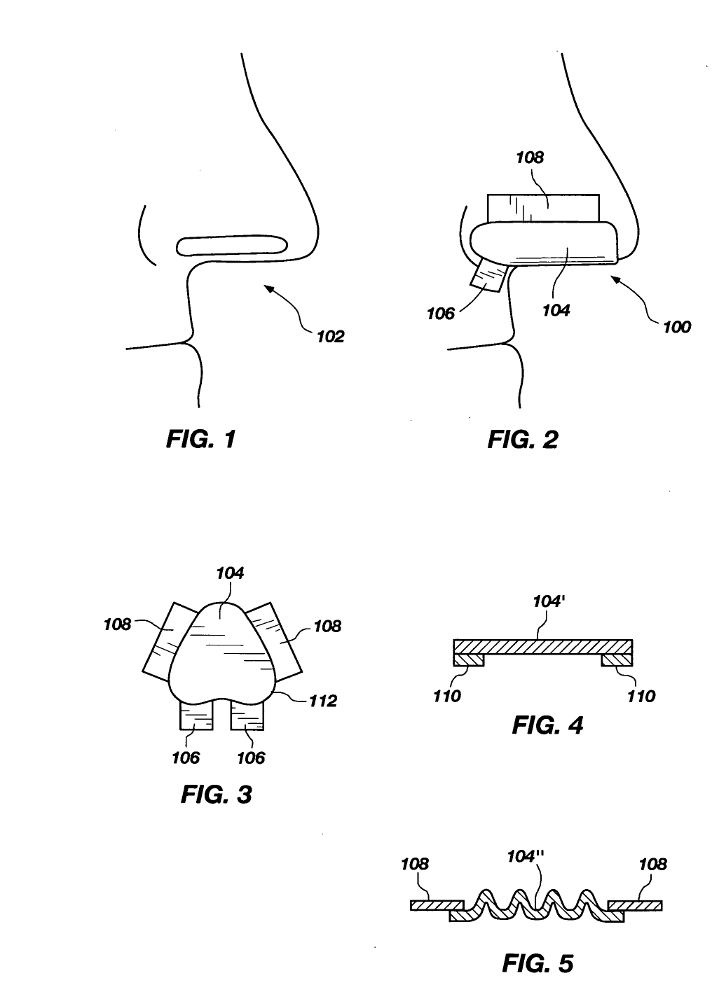

### DETAILED DESCRIPTION OF THE ILLUSTRATED EMBODIMENTS

A mini-nasal mask (generally indicated at 100 in FIG. 2) structured according to certain principles of the instant invention is adapted for its installation at the intake area, generally indicated at 102, of the nostrils of a human nose. In contrast to certain commercially available respiratory masks, a mini-nasal mask does not obscure the mouth of a user. Therefore, a user may eat, drink, and communicate with a lip-reader while wearing the mask.

Mask 100 includes a filter element 104 and an anchoring arrangement effective to hold the filter element in registration at the nasal intake area 102. An operable filter element is simply effective to strain particulate matter from air inspired through the nostrils. Various formulations of paper, or paper-like filters are workable, although the filter is not limited to such paper formulations. An alternative filter element 104 may include sponge-like or cloth-like material, and may be formulated from any effective natural or synthetic material, or combination of materials. One effective filter element 104 may even be formed from a portion of a commercially available face mask of the type typically disposed over a user's mouth and nose for use as a dust mask. Another operable filter element 104 may be made of polypropylene/polyethylene type materials such as may be found in a surgical/dental mask.

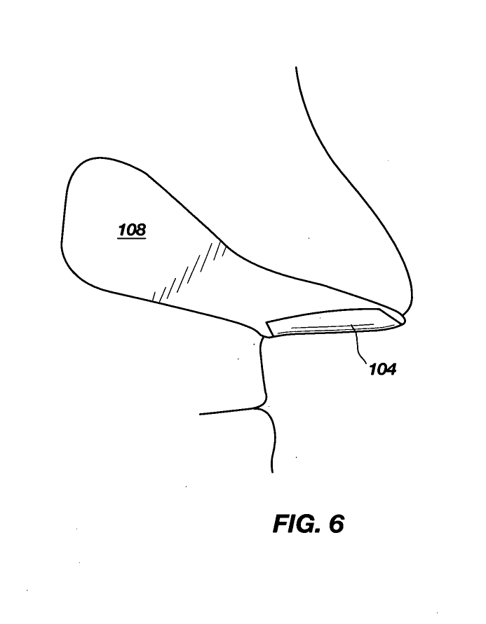

As illustrated in FIGS. 2 and 3, an operable anchoring arrangement includes a length of adhesive tape, such as lip tape 106 and/or nose/cheek tape 108. Tape 108 may be provided in different sizes and configurations, such as having an extended length compared to the illustration in FIG. 2, to permit anchoring to other facial structure, such as the user's cheek (see FIG. 6). A filter element may be affixed to a length of tape using adhesive, or other bonding mechanism, such as ultrasonic welding.

With continued reference to FIG. 6, it is within contemplation that a nasal mask within the ambit of the instant invention may be structured to substantially resemble a wound dressing, such as the ubiquitous Band-Aid™. In certain cases, one or more portion of the mask, such as the adhesive tape, may be substantially transparent or flesh-colored, to reduce its visible impact. However, in other cases, the mask may be structured to provide a striking and/or noticeable appearance. Another operable anchoring arrangement is illustrated in FIG. 4, where an equivalent adhesive tape may be formed by placing an adhesive element 110 on one or more perimeter portions of a filter element 104′.

It is within contemplation that an anchoring arrangement may include an elastic strap affixed to a filter element and structured for disposition of the strap to encircle a user's head, or arranged for attachment of the strap to a user's ear. It is further within contemplation that an anchoring arrangement may include a combination of adhesive, and/or adhesive tape, and/or elastic strap, and/or other structure operable to maintain the filter element in desired registration.

Inspiration of a breath through the nostrils can help to seat the filter element in registration against the external nares, thereby enhancing a seal between the filter element and nose. Desirably, the filter element will not impede flow of air to such an extent as to cause a user discomfort in breathing. Therefore, to promote air-flow, and to provide an extended operable life of a filter used in a dusty environment, the filter element may be enhanced, or arranged to provide an enlarged filtering surface area compared to the area defined by the nostril entrances. One such arrangement is illustrated in FIG. 5, where filter element 104″ includes a plurality of folds, corrugations, or pleated areas. A currently preferred enhanced filtering surface area is desirably larger, e.g. by a factor greater than about 1.2, than the area of a surface that would be defined by a membrane stretched from the boundary perimeter of the filtering surface area.

The filter element is desirably sized such that it can be entirely disposed within a boundary perimeter 112 (FIG. 3) sized in general agreement with a bottom of the nose. Typically, a boundary perimeter 112 of the filter element is smaller in size than the boundary perimeter of a 1½ inch square.

Sometimes, a filter element or other structure of a mini-nasal mask carries an aromatic cover scent, such as a flavoring, perfume, or other odiferous compound. Such filters may be used to advantage to cover unpleasant smells, such as when changing diapers, or during dressing changes on necrotic wounds. Certain filter elements may carry a bioactive agent, such as a compound of silver or an antibiotic, to resist transfer of germs, bacteria, viruses, and the like. A treatment compound may be applied to certain filter elements to impart nasal therapy while wearing a mask. A filter element may also be impregnated with a substance effective to enhance adherence of particles, such as pollen or dust, onto a surface of the filter element.

One filter element within contemplation is structured to permit a user to modify a size of the filtering surface area prior to installation of the mask. In such case, a one-size-fits-all mask may be provided, and each user may trim the filter element as required for comfort, or to fit their own respective nostril-opening area. In another embodiment within contemplation, the filter element is formed having sufficient flexibility to permit draping it around the nostrils for anchoring with the filter disposed partially along the side of the nose.

What is claimed is:

1. A nasal mask, comprising:
a filter element structured for disposition at, and external to, the intake area of the nostrils of a human nose; and
an anchor arrangement effective to hold said filter element in registration at said intake area.

2. The nasal mask of claim 1, wherein:
said filter element can be entirely disposed within a boundary sized in general agreement with a bottom perimeter of said nose.

3. The nasal mask of claim 1, wherein:
a perimeter of said filter element is smaller in size than a 1½ inch square.

4. The nasal mask of claim 1, wherein:
said filter element carries an aromatic cover scent.

5. The nasal mask of claim 1, wherein:
said filter element carries a bioactive agent.

6. The nasal mask of claim 1, wherein:
said filter element is impregnated with a substance effective to enhance adherence of particles onto a surface of said filter element.

7. The nasal mask of claim 1, wherein:
said filter element is configured to provide an enlarged filtering surface area compared to the area defined by the entrance to the nostrils of a human nose.

8. The nasal mask of claim 1, wherein:
said filtering surface area is larger, by a factor greater than about 1.2, than the area of a surface that would be defined by a membrane stretched from the boundary perimeter of said filtering surface area.

9. The nasal mask of claim 1, wherein:
said filter element is structured to permit a user to modify a size of the filtering surface area prior to installation of said mask.

10. The nasal mask of claim 1, wherein:
said anchor arrangement comprises a length of adhesive tape.

11. The nasal mask of claim 10, wherein:
said adhesive tape is structured to permit its attachment to an external surface of said nose.

12. The nasal mask of claim 10, wherein:
said adhesive tape is structured to permit its attachment to a human lip.

13. The nasal mask of claim 10, wherein:
said adhesive tape is substantially transparent.

14. The nasal mask of claim 10, wherein:
said adhesive tape is flesh-colored.

15. The nasal mask of claim 1, wherein:
said anchor arrangement comprises an elastic strap structured for disposition to encircle a user's head.

16. The nasal mask of claim 1, wherein:
said anchor arrangement comprises an elastic strap structured for attachment to a user's ear.

17. A nasal mask, comprising:
a filter element sized in general agreement with the intake area of a human nose; and
an adhesive anchor arrangement effective to hold said filter element in registration external to said nose and at said intake area.

18. The nasal mask of claim 17, wherein:
said anchor arrangement comprises first adhesive tape arranged to permit its adherence to the external surface of said nose and/or cheek.

19. The nasal mask of claim 18, further comprising:
second adhesive tape arranged to permit its adherence to the external surface of a user's lip.

20. A method for a user to filter inspired air, comprising:
adhesively anchoring a filter element to facial structure of said user effective to dispose said filter element external to the nose of said user, and in the path of inspired air, while maintaining unobstructed visibility to the mouth of said user for a bystander.
